(19) United States
(12) Patent Application Publication
(10) Pub. No.: US 2020/0129525 A1
(43) Pub. Date: Apr. 30, 2020

(54) Method to improve quality of life in non-primate animals by administration of Dehydroepiandrosterone Sulfate (DHEAS) sufficient to achieve primate-specific levels of circulating DHEAS that are not natural to the target species

(71) Applicant: Jonathan Wesley Nyce, Collegeville, PA (US)

(72) Inventor: Jonathan Wesley Nyce, Collegeville, PA (US)

(21) Appl. No.: 16/177,392

(22) Filed: Oct. 31, 2018

(51) Int. Cl. A61K 31/5685; A61K 9/00; A61P 25/28

(52) U.S. Cl. CPC A61K 31/5685; A61P 25/28; A61K 9/0056

(57) ABSTRACT

Canine quality of life, as judged by playfulness and sociability, rapidly declines as a dog ages. Dehydroepiandrosterone Sulfate (DHEAS) is an androgen that opposes cortisol in the fight or flight response, and we have proposed that wolves with exceptionally high circulating levels of DHEAS were the ones that interacted with humans, initiating the process of domestication that led to canis familiaris familiaris, the dogs we know today. Primates, particularly humans, have extremely high levels of circulating DHEAS. We discovered that administering primate levels of DHEA to canines dramatically increases their quality of life, as judged by remarkable increases in their playfulness and sociability. This discovery appears poised to maintain canine “puppy-like” behavior throughout the canine lifespan.

### STATEMENT REGARDING FEDERAL FUNDING

The inventor has been extensively funded by the National Cancer Institute, mostly in the field of cancer drug resistance. This funding does not appear related to the current invention. However, the inventor has received recent funding from the Food & Drug Administration, in the form of fee waivers, that is related to the current invention. The identifiers for this FDA funding are as follows: G003126-C-2195-SR1891; G003126-C-2195-SR1892; G003126-C-2195-5R1893; G003126-C-2195-SR1894; G003126-C-2195-SR1895; all to JWN.

### RELATED APPLICATIONS

EFS ID 29793860. Discovery of an adrenal androgen-kill switch that induces irreversible inhibition of Glucose-6-phosphate Dehydrogenase (G6PD) in cells experiencing inactivation of the TP53 tumor suppressor, and administration of dehydroepiandrosterone sulfate (DHEAS) to obtain levels of circulating DHEAS that optimize the function of this kill switch . . . .

### PRIOR DISCLOSURE

There has been no prior disclosure of this invention

### BACKGROUND OF THE INVENTION

### 1. Technical Field

This invention relates to veterinary drugs intended to improve quality of life for non-primate animals.

### 2. Description of Related Art

Quality of life drugs are part of the intellectual property landscape. For example, Bell (U.S. Pat. No. 6,165,504A) taught a method for improving the “quality of life” in castrated cancer patients using cyproterone acetate. Smith (U.S. Pat. No. 9,701,747B2) demonstrated that some drug treatments can improve both quality of life, and increase longevity of humans suffering from certain illnesses and exhibiting, or in danger of exhibiting C-reactive protein. In animals, Zicker et al (U.S. Pat. No. 8,496,981B2) and Yamka and Friesen (U.S. Pat. No. 8,148,325B2) both reported methods and compositions designed to improve the quality of life of a growing animal, specifically by the addition of omega-3 polyunsaturated fatty acids to foodstuff. Pitha et al (U.S. Pat. No. 8,563,522B2) described methods and compositions designed to maintain and/or attenuate a decline in quality of life by administering daily mannoheptulose to an animal.

### BRIEF DESCRIPTION OF INVENTION

It is a common experience that playfulness and sociability—major determinants of quality of life in pet animals—often decline as canines age, or after they are neutered, which generally happens by the end of their first year of life. This disappearance of playfulness and sociability (Early Onset Lethargy; EOL) represents a serious problem as it reduces quality of life for both the animal and its owner. If playfulness and sociability could be restored in aging and/or neutered pets with EOL, i.e., if quality of life could be restored, it would represent an important advance in veterinary medicine, increasing the quality of life for both the animal and its owner.

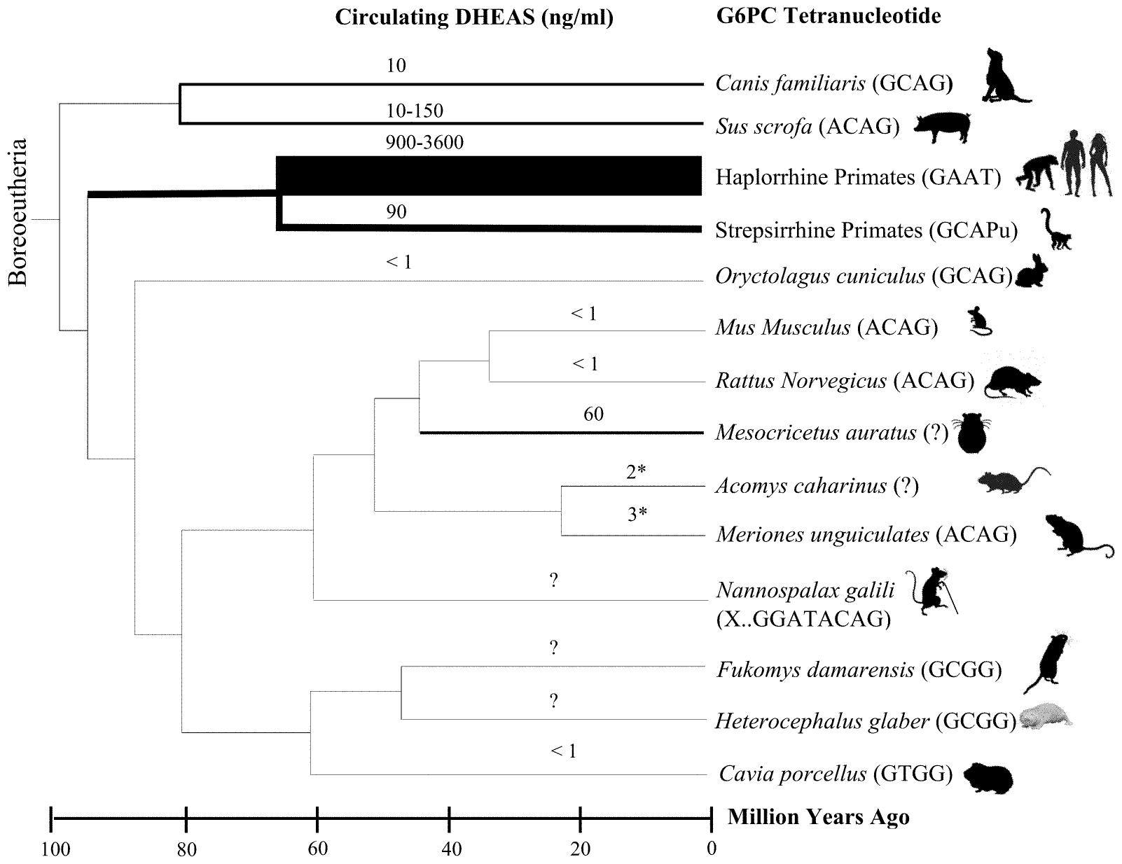

Canines are extremely unusual in the animal kingdom in having significant amounts of Dehydroepiandrosterone sulfate (DHEAS) circulating in their bloodstream. Circulating DHEAS is considered to be a feature of primates (FIG. 1). DHEAS functions as a precursor to DHEA because it can be imported into cells, desulfated, and act as a precursor to steroid hormones such as testosterone and estrone on an “as needed” basis; so called intracrinology (Labrie, 2018). DHEA is also known to oppose cortisol, the central mediator of the “fight or flight” response. We have proposed that the domestication of the wolf may have occurred when wolves with unusually high levels of circulating DHEAS came into contact with humans (Nyce, 2107). In such wolves, the inhibition of the fight or flight response by DHEA may have enabled closer contact with humans than would have been tolerated by wolves without high levels of circulating DHEAS. In other words, wolves with higher than normal levels of circulating DHEAS became domesticated, creating dogs. We further hypothesized that administration of supra-normal levels of DHEAS to canines suffering from EOL might restore playfulness and sociability in such animals, i.e., it may improve their quality of life.

Similarly, circulating levels of DHEAS are even lower in animals typically held in restrictive environments, such as zoos, or in the cattle and pork industries. Induction of supraphysiological levels of circulating DHEAS in these animals, too, will improve their quality of life by opposing stress hormones.

### DETAILED DESCRIPTION OF THE DRAWINGS

FIG. 1. Significant levels of DHEAS primarily occur only in primates; however, dogs have appreciable levels, too, encouraging the inventor to hypothesize that above average levels of circulating DHEAS in some wolves enabled their domestication; and further, that administration of supra-physiological doses of DHEA to dogs may restore playfulness and sociability in dogs that have suffered a decrement in these aspects of their quality of life.

Table 1. Results of a study on 60 dogs demonstrating a remarkable improvement in quality of life, as measured by owner estimation of playfulness and sociability before and after treatment. Playfulness was defined as a dog initiating solitary play. Sociability was defined as a dog initiating play with another pet or with its owner. Remarkably, fifty-six of sixty dog owners reported a clear increase in their dogs playfulness and/or sociability during the treatment period. Of twenty-six dogs treated the highest doses (25 or 50 mg/kg) eighteen owners reported the changes in their dogs to be dramatic.

### DETAILED DESCRIPTION OF THE INVENTION

Physiological levels of circulating DHEAS in canines are 10 ng/ml (±2 ng/ml) (Frank et al, 2003; Odell & Parker, 1985; Tremblay & Belanger, 1985; Mialot et al, 1988; Mongillo et al, 2014; Rondelli et al, 2015). Our invention employs doses of DHEAS that produce supra-physiologic concentrations of circulating DHEAS at least nine times higher than this, i.e., at least 90 ng/ml, i.e. equivalent to the lower range of primate-specific high levels of circulating DHEAS (FIG. 1). This equates to a minimum treatment dose of 0.009 mg/kg. Our studies reported here were performed with DHEAS administered at ranges from 0.5-50 mg/kg. For all non-primate animals, this invention employs supra-physiologic doses of DHEAS that produce concentrations of circulating DHEAS that are at least ten-fold higher than those found naturally

We observed a profound increase in both playfulness (e.g., the initiation of solitary play with a toy or ball) and sociability (seeking playful interaction with owner or another pet animal) when DHEAS was administered at doses of 5 mg/kg and above; an effect could be observed even at the lower dose of 1 mg/kg, but it was not as no pronounced. Thus, in 56 of 60 treated dogs, owners reported observing a very clear improvement in their dog's playfulness and sociability, an observation that in most cases was also remarked upon by visitors to the owner's home. The uniformity of this finding is surprising, because the natural levels of canine DHEAS are quite low, and so this species has never been exposed to such high levels as we report here. Clearly, dogs experience an unmistakable positive response to exogenous DHEAS. Other species of companion animals such as cats, are expected to react similarly to exogenous DHEAS, but we have not yet performed such experiments.

We administered our DHEAS orally, as a liquid, or in capsules. A preferred embodiment of this invention are dosage formulations that mask the bitter flavor of DHEAS from dogs or other pet animals receiving it orally. As an example, Onda et al. (U.S. Pat. No. 4,385,078A) teach methods for providing enteric coatings for solid dosage forms. Tai (U.S. Pat. No. 5,013,557A) teaches taste masking compositions comprising spray dried microcapsules containing sucralfate, and Alkire (U.S. Pat. No. 5,607,697A) and Cherukuri et al (U.S. Pat. No. 5,013,716A) teach the manufacture of taste masking microparticles for oral dosage forms. Patell (U.S. Pat. No. 4,916,161A) teaches the use of taste-masking pharmaceutical agents via wet granulation. In short, many methods are available to produce a formulation that will effectively prevent dogs or other pet animals from experiencing the bitter taste of DHEAS. Very little work has been done using DHEAS as the pharmaceutical ingredient, with most studies utilizing DHEA. For example, US20140235598A1 teaches the manufacture of tablets containing DHEA, but a similar formulation could be rendered with DHEAS.

DHEAS can most conveniently be formulated as pellets or micropellets that could be added to food according to the body weight of the animal, such formulations designed to be neutral in flavor, or flavored according to the palate of the target animal, but in either case masking the bitter taste of DHEAS. In another embodiment of this invention, the DHEAS micro-pellets provide such a stabilized formulation for DHEAS that they can be pre-added to food, such that a specific amount of said food is then administered to the target animal, according to the animal's weight. Alternatively, DHEAS can be formulated such that it can be added to the target animal's drinking water, either as a liquid, or as pellets, crystals or powder that will dissolve in water.

DHEAS could also be administered as a transdermal. Riepl (US20130231317A1) teaches the formulation of a DHEA bioadhesive controlled release gel. Rosenbaum (U.S. Pat. No. 5,869,090A) teaches the transdermal delivery of DHEA by additional means.

Fields (US20150209399A1) teaches the use of green coffee bean extract to enhance the levels of natural dehydroepiandrosterone.

DHEAS can be synthesized by any number of methods. For example, Sobel et al developed methods employing reaction of the parent steroid with sulfate salts and acetic anhydride (Sobel et al 1936). Fiesler developed a method of steroid sulfation employing chlorosulfonic acid (Fiesler, 1948). A number of groups have developed methods for sulfation of steroids employing amine complexes of sulfur trioxide (Sobel et al, 1941; Dusza et al 1968; 1985 a,b). Mumma developed a method employing sulfuric acid and carbodiimides (Mumma 1966). Joseph et al developed a method utilizing sulfamic acid (Joseph et al, 1966). Two groups developed methods employing sulfuryl imidazolium salts (Desoky et al, 2011; Liu et al, 2004). Waller and McLeod recently developed a simplified method of steroid sulfation which has the advantage of rapid purification by solid phase extraction (Waller and McLeod, 2014). With respect to synthesis of the parent steroid, DHEA, Hershberg (U.S. Pat. No. 2,656,364A, 1953) taught a method comprising reaction of dehydropregnenolone acetate oxime with toluene sulfonyl chloride, and heating the product with ethylene-diamine.

With respect to prior art related to treatments employing DHEAS, as noted above, most studies have utilized DHEA, not DHEAS. Garcia et al. (U.S. Pat. No. 9,066,956B2) teach a method comprising DHEA or DHEAS formulated within a vaginal ring for the treatment of symptoms related to menopause. Schwartz (U.S. Pat. No. 6,552,010) teaches the use of DHEA in the treatment of Systemic Lupus Erythematosus. Yen and Berger (U.S. Pat. No. 5,861,391A) teach the use of DHEA to treat primary adrenal insufficiency and Addison's disease in people with HIV infection. Labrie (US20110312925) teaches the use of DHEA in the treatment or prevention of estrogen-related diseases. Zenk (U.S. Pat. No. 7,199,116B2) teaches the use of analogs of DHEA to increase metabolic rate in dieting mammals. White and Gyurik (U.S. Pat. No. 8,883,769B2) teach methods for the treatment of fibromyalgia and chronic fatigue syndrome by compositions of androgens. Labrie (U.S. Pat. No. 5,846,950A) teaches methods for preventing and treating osteoporosis with low dose non-masculinizing androgenic compounds. In short, a variety of applications have been proposed for the use of DHEA in human diseases. However, none appear germane to our intended use as a method to restore playfulness and sociability in pet dogs.

A critical feature of this invention is that it must consist of a dosage formulation that masks the bitter flavor of DHEA from dogs or other pet animals receiving it orally. As an example, Onda et al. (U.S. Pat. No. 4,385,078A) teach methods for providing enteric coatings for solid dosage forms. Tai (U.S. Pat. No. 5,013,557A) teaches taste masking compositions comprising spray dried microcapsules containing sucralfate, and Alkire (U.S. Pat. No. 5,607,697A) and Cherukuri et al (U.S. Pat. No. 5,013,716A) teach the manufacture of taste masking microparticles for oral dosage forms. Patell (U.S. Pat. No. 4,916,161A) teaches the use of taste-masking pharmaceutical agents via wet granulation. In short, many methods are available to produce a formulation that will effectively prevent dogs or other pet animals from experiencing the bitter taste of DHEA.

In addition to the oral and transdermal methods described above, DHEA or its sulfated form DHEAS can be injected as a long-acting depot. For example, Labrie and Lepage (U.S. Pat. No. 5,434,146A) teach methods to deliver timed-release biodegradable microparticles for long-lasting delivery of steroids.

### U.S. PATENTS CITED

Bell R G U.S. Pat. No. 6,165,504A 2000 Grant, expired. Methods for treating hot flashes and improving the quality of life of castrated prostatic cancer patients.
Zicker S C et al U.S. Pat. No. 8,496,981B2 2013 Grant. Methods for enhancing the quality of life of a growing animal.
Smith J T L U.S. Pat. No. 9,701,747B2 2017 Grant. Method of improving patient survivability and quality of life by anti-IL-6 antibody administration.
Yamka R, Friesen K G U.S. Pat. No. 8,148,325B2 2012 Grant. Methods for enhancing the quality of life of a senior animal.
Pitha J et al. U.S. Pat. No. 8,563,522B2 2013 Grant. Method of maintaining and/or attenuating a decline in quality of life.
Garcia, Frans Ariel Fuentes, et. al., U.S. Pat. No. 9,066,956B2 priority date 2011 Dec. 29, Vaginal ring comprising DHEA or DHEA sulfate and optionally a release-modulating agent of the active principle, useful to increase the ovarian reserve in women and to relieve symptoms associated with menopause.
Herman Jan Tijmen Coelingh Bennink et al., US20140235598A1 priority date 2011 Jul. 19 Tablet containing dehydroepiandrosterone.
Riepll, M S, US20130231317A1 priority date 2011 Jul. 19 DHEA bioadhesive controlled release gel.
Hershberg, Emmanuel B, U.S. Pat. No. 2,656,364 1953 Grant Process for the manufacture of 17-keto steroids.
Schwartz, K E, U.S. Pat. No. 6,552,010 2003 Grant, Treatment of SLE with dehydroepiandrosterone.
Yen, S S C and Berger, B, U.S. Pat. No. 5,861,391A priority 1997 Jan. 29, Use of dehydroepiandrosterone to treat primary adrenal insufficiency and Addison's disease.
Labrie, F, US20110312925 priority 2010 Jun. 16, Methods of treating or preventing estrogen-related diseases.
Zenk, J, U.S. Pat. No. 7,199,116B2 Priority 2003 Jan. 13, Method of modulating the basal metabolic rate of a dieting mammal by administration of a metabolic modulating agent to the dieting mammal.
Rosenbaum, J, US586909A granted 1999, Transdermal delivery of dehydroepiandrosterone.
White, H D and Gyurik, R, U.S. Pat. No. 8,883,769B2 priority 2003, Methods for the treatment of fibromyalgia and chronic fatigue syndrome.
Fields, C C, US20150209399A1 priority 2013, Methods for enhancement of dehydroepiandrosterone using green coffee been extract.
Labrie, F, U.S. Pat. No. 5,846,950A priority 1991, Methods for preventing and treating osteoporosis with low dose non-masculinizing androgenic compounds.
Onda, Y et al., U.S. Pat. No. 4,385,078A Priority 1978, Method for providing enteric coating on solid dosage forms and aqueous compositions therefor.
Alkire, T G, U.S. Pat. No. 5,607,697A 1995, Taste masking microparticles for oral dosage forms.
Cherukuri, S R et al, U.S. Pat. No. 5,013,716A 1988, Unpleasant taste masking compositions and methods for preparing same.
Tai, A W, U.S. Pat. No. 5,013,557A 1989, Taste masking compositions comprising spray dried microcapsules containing sucralfate and methods for preparing same.
Labrie, F, and Lepage, M. U.S. Pat. No. 5,434,146A 1991, Controlled release systems and low dose androgens.

### NON PATENT LITERATURE CITED

Ashley P F, Frank L A, Schmeitzel L P, Bailey E M, Oliver J W 1988 Effect of oral melatonin administration on sex hormone, prolactin, and thyroid hormone concentrations in adult dogs. Journal American Veterinary Medical Association 215(8):1111-1115.
(https://www.ncbi.nlm.nih.gov/pubmed/?term=Effect+of+oral+melatonin+administration+on+sex+hormone%2C+prolactin%2C+and+thyroid+hormone+concentrations+in+adult+dogs)
Desoky A Y, Hendel J, Ingram L, Taylor S D 2011 Preparation of trifluoroethyl- and phenyl-protected sulfates using sulfuryl imidazolium salts. Tetrahedron 67:1281-7.
Dusza J P, Joseph J P, Bernstein S. 1968 Steroid conjugates IV. The preparation of steroid sulfates with triethylamine-sulfur trioxide. Steroids 12:49-61.
Dusza J P, Joseph J P, Bernsteins S 1985a The preparation of estradiol-17beta sulfates with triethylamine-sulfur trioxide. Steroids 12:49-61.
Dusza J P, Joseph J P, Bernstein S 1985b A fusion method for the preparation of steroid sulfates Steroids 45:317-23.
Fielsler L F 1948 Napthoquinone antimalarials. XVI. Water soluble derivatives of alcoholic and unsaturated compounds. J Am Chem Soc 70:3232-7.
Frank L A, Rohrbach B W, Bailey E M, West J R, Oliver J W 2003 Steroid hormone concentration profiles in healthy intact and neutered dogs before and after cosyntropin administration. Domestic Animal Endocrinology 24(1):43-57.
(https://www.ncbi.nlm.nih.gov/pubmed/?term=Steroid+hormone+concentration+profiles+in+healthy+intact+and+neutered+dogs+before+and+after+cosyntropin+administration)
Joseph J P, Dusza J P, Bernstein S 1966 Steroid conjugates I. The use of sulfamic acid for the preparation of steroid sulfates. Steroids 7:577-87.
Labrie, F. (2018) Intracrinology and menopause: the science describing the cell-specific intracellular formation of estrogens and androgens from DHEA and their strictly local action and inactivation in peripheral tissues. Menopause. 2018 Aug. 20. doi: 10.1097/GME.0000000000001177. [Epub ahead of print]
Liu Y, Lien I-F F, Ruttgaizer S, Dove P, Taylor S D 2004 Synthesis and protection of aryl sulfates using 2,2,2-trichloroethyl moiety. Org Lett 6:209-12
Mialot J P, Thibier M, Toublanc J E, Castanier M, Scholler R 1988 Plasma concentration of luteinizing hormone, testosterone, dehydroepiandrosterone, androstenedione between birth and one year in the male dog: longitudinal study and hCG stimulation. Andrologia 20(2):145-54.
(https://www.ncbi.nlm.nih.gov/pubmed/?term=Plasma+concentration+of+luteinizing+hormone%2C+testosterone%2C+dehydroepiandrosterone%2C+androstenedione+between +birth+and+one+year+in+the+male+dog%3A+longitudinal+study+and+hCG+stimulation)
Mongillo P, Prana E, Gabai G, Bertotto D, Marinelli L 2014 Effect of age and sex on plasma cortisol and dehydroepiandrosterone concentrations in the dog (Canis familiaris). Research in Veterinary Science 96(1):33-8.
(https://www.ncbi.nlm.nih.gov/pubmed/?term=Effect+of+age+ and+sex+on+plasma+cortisol+and+dehydroepiandrosterone+concentrations+in+the+dog)
Mumma R O 1966 Preparation of sulfate esters. Lipids 1:221-3.
Nyce J W 2017 Species-specificity of an adrenal androgen-mediated kill switch that is triggered by the inactivation of the p53 tumor suppressor. Translational Medicine Reports 1(3):76-85 http://www.pagepressjournals.org/index.php/tmr/article/view/6773
Odell W D, Parker L N 1985 Control of adrenal androgen production. Endocrine Research 10(3-4):617-630. https://www.ncbi.nlm.nih.gov/pubmed/6100259
Rondelli M C, Munhoz T D, Catandi P B, Freschi C R, Palacios Junior R J, Machado R Z, Tinucci-Costa M 2015 Serum DHEA-S increases in dogs naturally infected with Ehrlichia canis. Research in Veterinary Science 100:18-20.
(https://www.ncbi.nlm.nih.gov/pubmed/?term=Serum+DHEA-S+increases+in+dogs+naturally+infected+with+Ehrlichia+canis)
Schiebinger R J, Albertson B D, Barnes K M, Cutler G B Jr, Loriaux D L 1981 Developmental changes in rabbit and dog adrenal function: a possible homologue of adrenarche in the dog. American Journal of Physiology 240(6):E694-9.
(https://www.ncbi.nlm.nih.gov/pubmed/6264794)
Sobel A E, Drekter I J, Natelson S. 1936 Estimation of small amounts of cholesterol as the pyridine cholesteryl sulfate, J. Biol Chem. 115:381-90.
Sobel A E, Spoerri P E 1941 Steryl sulfates. I. Preparation and properties. J Am Chem Soc 63:1259-61.
Tremblay Y, Belanger A 1985 Changes in plasma steroid levels after single administration of hCG or LHRH agonist analogue in dog and rat. Journal of Steroid Biochemistry 22(3):315-320. (https://www.ncbi.nlm.nih.gov/pubmed/2581068)
Waller C C, McLeod M D (2014) A simple method for the small scale synthesis and solid phase extraction purification of steroid sulfates. Steroids 92:74-80.
https://core.ac.uk/download/pdf/156626558.pdf

That which is claimed is:

1. A method of restoring playfulness and sociability in dogs and other non-primate animals by administration of supra-physiologic doses of Dehydroepiandrosterone Sulfate (DHEAS), i.e., by “primatizing” them with respect to circulating DHEAS.

2. The method in claim 1 in which the DHEAS formulation is modified so as to mask its bitter taste.

3. The method in claim 1 in which the DHEAS is administered in a timed-release formulation.

4. The method in claim 1 in which DHEAS is administered orally, as beads or microbeads, in pellets, micro-pellets, capsules, tablets, or as a liquid, including a liquid formulated to have flavor and olfactory characteristics favorable to the target species, or in food directly or formulated in any of the noted ways.

5. The method in claim 1 in which DHEAS is administered at a dose of, preferentially, 0.05 to 100 mg/kg; or more preferentially, 0.5 to 75 mg/kg; or most preferentially, 5-25 mg/kg.
